(19) United States
(12) Patent Application Publication
(10) Pub. No.: US 2004/0073455 A1
(43) Pub. Date: Apr. 15, 2004

(54) Child care telehealth access network

(75) Inventors: Kenneth M. McConnochie, Pittsford, NY (US); Nancy Wood, Macedon, NY (US); Neil Herendeen, Pittsford, NY (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: University of Rochester Medical Center, Rochester, NY

(21) Appl. No.: 10/435,601

(22) Filed: May 8, 2003

Related U.S. Application Data
(60) Provisional application No. 60/379,122, filed on May 8, 2002.

(51) Int. Cl. G06F 17/60; A61B 5/00

(52) U.S. Cl. 705/2; 600/300

(57) ABSTRACT

A system and a method for providing medical evaluation and treatment recommendations for children at a childcare center, including: an examination station for medically examining a patient, located at the childcare center, wherein the examination station includes an examination computer, an examination camera coupled with the computer, and a diagnostic tool set coupled with the computer; a diagnosis station for diagnosing the condition of the patient, remotely located from the examination station; wherein the diagnosis station includes a diagnosis computer, a diagnosis camera coupled with the diagnosis computer; modules for interfacing the diagnosis computer with the examination computer; and a communication system for coupling the examination station with the diagnosis station.

### CROSS-REFERENCES TO RELATED APPLICATIONS

This application claims priority to U.S. Patent Application No. 60/379,122, filed May 8, 2002, the disclosure of which is hereby incorporated by reference in its entirety for all purposes.

### STATEMENT AS TO RIGHTS TO INVENTIONS MADE UNDER FEDERALLY SPONSORED RESEARCH OR DEVELOPMENT

The research that led to this invention was partly carried out with Government support under Contract No. 36 60 100021 awarded by the U.S. Department of Commerce Technology Opportunities Program. The Government has certain rights in the invention.

### BACKGROUND OF THE INVENTION

The present invention relates, generally, to systems and methods for the remote examination, diagnosis and treatment of patients, by health care providers who are not co-located with their patients.

Tremendous socioeconomic disparities in childhood morbidity burden persist. Some research comparing hospitalization rates for impoverished, inner city, children with those of their more affluent suburban counterparts documents striking differences. For example, one research shows that inner city children had a 5-fold greater rate for asthma exacerbation, nearly a 4-fold greater rate for bronchiolitis, and a 3-fold greater rate for gastroenteritis/dehydration. Although differences in physical environment (e.g., environmental tobacco smoke, household crowding) may account for some of the socioeconomic disparity, treatment which is delayed or less appropriate also is likely to be an important etiologic factor.

Less effective treatment is closely tied to difficulties in access to care. Inner city children not only endure a greater burden of morbidity, but their families have less social, material and financial resources to address this burden. Limitations in transportation and communication resources are critical for health care access. For example, data based on the 1990 US Census results for Rochester, N.Y., shows that 42% of Rochester's inner city households had no automobile and 13% lacked a phone. These figures compared with 4.6% (no automobile) and 0.7% (no phone) for suburban households. Generally, inner city families are served by health centers and hospital-based clinics whose strained resources can provide only limited continuity of care and limited evening office hours, as well as inconsistent and slowly responding phone coverage systems. Use of hospital emergency departments to address problems that could often be managed by phone, or office visits is a frequent consequence.

Care outside the home has become the norm for pre-school children in the United States. Already in 1995, 60% of children from birth to 5 years of age, who were not yet in school, participated in a non-parental childcare or early education program. With continuation of the trend for young mothers to join the work force and the advent of welfareto-work programs throughout the US, this proportion is undoubtedly larger today.

Acute, generally infectious illness is a very common and difficult problem for all involved in childcare centers. Higher incidence and greater severity of illness among children in childcare than among children in home care is well documented. Economic burden of illness in day care is also substantial. Direct and indirect costs are attributed primarily to costs of physician visits, medications, alternative care arrangements and parent time lost from work.

Childcare centers have the difficult responsibility of determining whether illnesses require the child to be removed from the center and sent to a physician. Some of the regulations relating to health in childcare are only stated in general terms despite thoughtful efforts that have gone into their development. They are subject to judgment, various interpretations and legitimate debate. Developing the operational details of exclusion policies is left to individual day care centers. Policies often require an MD visit to certify readiness for return to childcare.

Parents find themselves in an even more difficult situation. They are frequently called at their jobs to pick up ill children. One study found that a child's illness accounted for 40% of missed work for childcare parents. Inner city parents may jeopardize employment by leaving work as demanded. Other parents, anxious to keep jobs that they can't afford to lose, try to delay ill child pick-ups and hasten the return of children to the center for care. Centers are pressured by parents to interpret exclusion policies loosely. Some childcare centers' leadership report that febrile illness frequently becomes apparent late in the morning, at the time that antipyretic medication, given by parents shortly before morning drop-off time, wears off. Nurses and physicians whose children use childcare report informally that they have often resorted to this practice. Compared to a professional and middle class parent, however, an inner city parent is less likely to have flexibility in work hours, a spouse with flexible work hours, and ready access to a health care provider for diagnosis, treatment, guidance and certification that will allow rapid return to childcare.

To summarize the problem, acute medical problems, generally infectious in nature, commonly challenge all involved in childcare. Resources for dealing with this burden of illness are not readily available to many impoverished, inner city families. Thus, the children and families most burdened by illness, and whose reliance on day care is most important to their ability to improve economic circumstances, are those least equipped to deal with the challenge.

There is therefore a need to improve the access of children in day care centers to effective health care services, while minimizing the burden of providing such a service to their primary caregivers.

### BRIEF SUMMARY OF THE INVENTION

The present invention provide a system and a method for providing medical evaluation and treatment recommendations for children at a childcare center, including: an examination station for medically examining a patient, located remotely from a physician, wherein the examination station includes an examination computer, an examination camera coupled with the computer, and a diagnostic tool set coupled with the computer; a diagnosis station for diagnosing the condition of the patient, remotely located from the patient; wherein the diagnosis station includes a diagnosis computer, a diagnosis camera coupled with the diagnosis computer; modules for interfacing the diagnosis computer with the examination computer; and a communication system for coupling the examination station with the diagnosis station.

One aspect of the present invention is directed to a method for providing medical evaluation and treatment recommendations for children at a childcare center, including: providing an examination station at a childcare center; providing a diagnosis station at a location different from the examination station; providing a communication interface between the examination station and the diagnosis station; examining a patient using the examination station to generate examination data; transmitting the examination data to the diagnosis station; evaluating the condition of the patient using the diagnosis station to generate evaluation data; and communicating the evaluation data from the diagnosis station to the examination station using the interface.

These and other embodiments of the present invention, as well as its advantages and features, are described in more detail in conjunction with the description below and attached figures.

### BRIEF DESCRIPTION OF THE DRAWINGS

FIG. 1 is a block diagram of an embodiment of a system for the provision of health care services by a health care provider at a remote location to a patient at a childcare center, in accordance with the present invention.

FIG. 2 is a block diagram showing an embodiment of a network model between a day care center and a clinician site in accordance with the present invention.

FIG. 3 is a block diagram of an embodiment of an expanded version of the network model of FIG. 2.

FIG. 4 is a block diagram of an embodiment of a method for the provision of health care services by a health care provider at a remote location to a patient at a childcare center, in accordance with the present invention.

FIG. 5A is diagram of one embodiment of a computer system for executing a software program incorporating aspects of the described method for the provision of health care services by a health care provider at a remote location to a patient at a childcare center, in accordance with the present invention.

FIG. 5B is a simplified system block diagram of a typical computer system used to execute a software program incorporating aspects of the described method for the provision of health care services by a health care provider at a remote location to a patient at a childcare center, in accordance with the present invention.

### DETAILED DESCRIPTION OF THE INVENTION

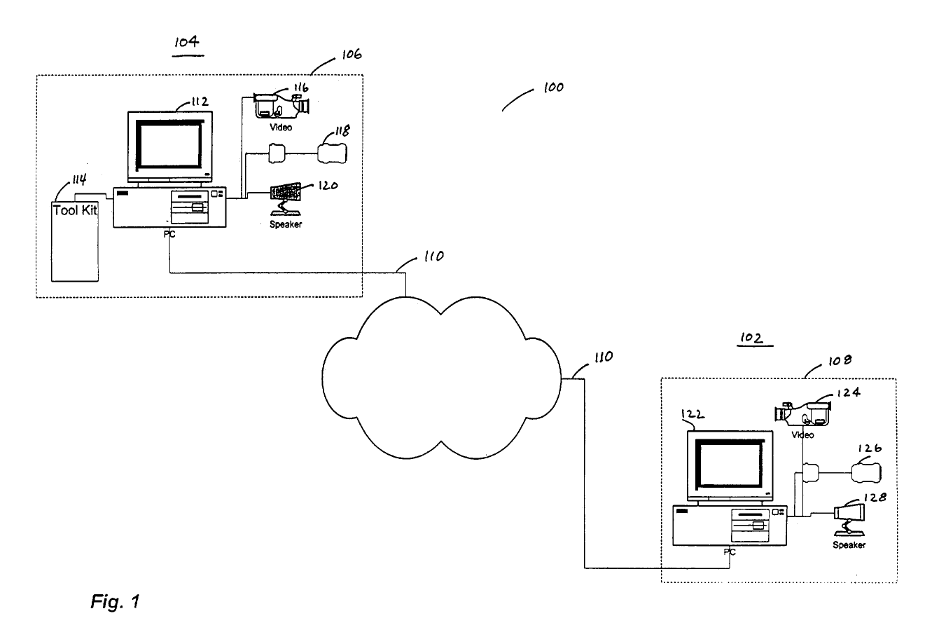

Embodiments of the present invention are directed towards methods and systems for the provision of health care services by a health care provider at a remote location to a patient at a childcare center. FIG. 1 is a block diagram of an embodiment of a system 100 for the provision of health care services by a health care provider at a remote location 102 to a patient at a childcare center 104, in accordance with the present invention. The system 100 includes, generally, a patient-side examination station 106, a clinician-side diagnostic station 108, a high speed connection 110 between the patient-side and the clinician-side diagnostic stations, as well as additional software and hardware for enabling the high speed connections as well as the interfacing of various input and output devices to each of the examination and diagnostic stations.

The examination station 106 is typically located at a facility where the patients are located such as, for example, a day care center. The examination station includes a computer such as, for example, a personal computer 112. Various diagnostic equipment 114 as well as various peripheral devices are interfaced with the computer 112. The peripheral devices include a camera (e.g., digital video or still camera) 116, a microphone 118 and speaker(s) 120. The diagnostic equipment include a telemedicine tool kit 114, which typically includes (not shown) a stethoscope 114a, a digital otoscope or a digital endoscope 114b, a thermometer 114c, an ophthalmoscope 114d, and other medical diagnostic devices as are deemed necessary by the unique medical needs of the patients to send diagnostic data including images or audio to the treating physician. Each of the devices in the tool kit 114 is interfaced with the computer 112. For example, the stethoscope is coupled with the computer 112, such that the sounds picked up by the stethoscope are played back at the speakers 120 connected with the computer 112. Likewise images picked up by the otoscope or endoscope 114b may be displayed on the display device of the computer 112. In like manner, the temperature, blood pressure, etc. of the patient are also available for display on the display device of the computer 112. Various hardware and or software devices are also configured to enable the interfacing of the various peripheral and telemedicine tool kit devices with the computer 112. Additionally, the computer 112 or the examination station 106 include modules for connecting to a high speed connection 110 for ultimately connecting to other computers such as, for example, the computer 122 at the clinician-side station 108 at the remote location 102.

The clinician-side diagnostic station 108 include a computer 122 and various peripheral devices that are coupled to the computer 122. The clinician-side peripheral devices include, for example, a video camera 124, microphone 126 and speakers 128. Additionally, the clinician side computer is able to communicate with the patient-side computer via a connection with the high speed connection 110. Furthermore, the clinician side computer 122 includes various hardware and/or software devices that are configured to enable the interfacing of the various peripheral devices at the clinician-side computer and those including the telemedicine tool kit devices 114 of the patient-side computer 112 with the computer 122.

Further details of an implementation of the system 100 are as set forth below. For the sending unit, for example the unit located at the childcare or day care center, the equipment setup includes a modern personal computer such as a 2000 MHz computer having 512 Mb of RAM; a 40 Gb HD; a 40×12×40 CDRW; a mid tower 350 watt case, a 32 Mb video card; a 1.44 floppy drive; a sound card; standard keyboard, speaker(s) and mouse; a video capture card; a color digital (e.g., CCD) camera; a microphone; a 17″ Flat tube 0.25 Monitor; appropriate operating system and productivity software (e.g., Windows 2000 pro and Office 2000); an otoscope and/or endoscope such as the commercially available Dr. Camscope; and electronic stethoscope; appropriate analyzer software (for use with electronic stethoscope); additional software such as the commercially available 2nd Opinion software; and an appropriate CODEC (e.g., Zydacron Z360) and Video Conferencing Software. For the receiving unit, for example the unit located at the clinician site, the equipment setup includes a setup similar to the setup used for the sending unit.

One implementation of the connection 110 connecting the sending and receiving units is as set forth below. The connection includes a DSL or better connection and a server. In this embodiment, the server setup includes a DataHive Server; a 80 GB RAID1 hot swappable disk storage; a 1 GHz Pentium III Processor; 512 MB of memory; 2 Intel Network Connections, where the DataHive Server features include: a Web Server, a proxy server, with HTTP support with CGI Scripting, Perl, PHP and SSL Support via Apache Web Server, an E-mail Server (e.g., SMTP, POP3, Domain Access Control), a DNS server with support for on-demand resolving, caching, master hosting and slave hosting, a DHCP server and client with PPPoE support, MySQL server, an IP Router, a common Standard Network Firewall, an Auto Resource Discovery for Firewall and Network Address Translation, a Network Address Translation with Port Mapping, an FTP Server, as well as VPN-PPTP and IPSec protocols.

With such a system in place, a health care provider at the remote location 102 is enabled to make a virtual house call at the day care site 104. In practice, a sick child is examined at the daycare site by a trained telehealth assistant who is using the patient-side examination station. Training of a lay-person to serve as a telehealth assistant can usually be accomplished in two weeks following a training protocol developed by the inventors herein. To successfully complete the training process, competency must be demonstrated on key tasks integral to the remote examination. The telehealth assistant may also be a nurse, but whether the person is or not is inconsequential, the person is primarily a telehealth assistant trained to use the examination station and provide the relevant information to the clinician at the diagnosis station. The training of a lay person to serve as a telehealth assistant makes it possible to serve more childcare programs and be more cost-effective. The relevant information is transmitted in a nearly real-time manner to the clinician at the clinician site, who makes appropriately diagnoses of the patient's condition with the aid of the information, including audio, voice and video data in consultation with the telehealth assistant and the patient.

For example, as the telehealth assistant (at the day care site) inserts the otoscope into a child's ear, doctors (at the remote clinic) are able to see in nearly real time, streaming video what they would if the child came to their office. Likewise, as the stethoscope is placed on the child, doctors can listen to the child's breathing or his or her stomach gurgling, via the speaker(s) 128 coupled to the diagnostic station's computer 122. Similarly, a camera 116 helps diagnose illnesses such as skin disorders, by displaying the images captured by a telehealth assistant at the day care site on the display device of the computer 122 at the clinician's site. Additionally, the microphone 126 and video camera 124 at the clinician station 108 are used to capture the doctor's voice and image and display them on the display device of the computer 112 at the patient's site. The doctor is thus enabled to speak with the telehealth assistant and the patient and interactively diagnose the condition of the patient. Thus, a system 100 in accordance with embodiments of the present invention enables a doctor to nearly do everything a doctor would normally do, except touch the child, and for that the doctors at the remote site may rely on a nurse at the day care site to be their hands. Using the system 100, the pediatrician can usually determine whether a child with symptoms needs to sent home from the center or needs to see a physician in person. Results of initial experimental use of the system indicate that more than 95 percent of the time diagnosis and treatment decisions can be made on the basis of the telemedicine evaluation alone. During an initial experimental use of the system, the most common reasons for the system's use in a childcare of day care environment were for minor acute problems such as colds with fever, ear infections, pink-eye, and impetigo.

Furthermore, in an effort to maintain full communication and to provide as complete a medical history as possible, a report of the diagnosis and prescribed treatment is sent (e.g., electronically or via fax) to the child's regular health care provider. A detailed post-visit instruction sheet is also provided to the parent or guardian when the child is picked up at the daycare center. Doctors have the ability to capture high-resolution images—for instance, a digital images of a child's middle ear—and e-mail them to their colleagues or the child's health care provider for a second opinion. Once a diagnosis is made, doctors can call in a prescription to the pharmacy of the patient's choice for delivery to the patient at the day care center. In addition to the calling in of prescription, and the reporting to the regular health care provider(s), such follow-on work may also include the forwarding of the relevant information to an insurance provider. Such follow-on work data can also be shared amongst the various sites using similar hardware setups located at the various sites, which are also enabled to communicate with each other using a network connection 110.

Various computer software and/or hardware devices are used at both the patient-side and the clinician-side computers to enable the interfacing, data acquisition, data processing, and the transmitting and receiving of various data by the patient-side and the clinician-side computers, examples of which have been set forth above. Other arrangements are known to those skilled in the arts of interfacing computers and/or communications equipment.

One aspect of the telehealth system in accordance with embodiments of the present invention is the high-speed data network that enables the flow of data between the patient-side and clinician-side computer-bases stations. A high-speed network allows for the nearly real-time communications between the patient/telehealth assistant and the doctor, including telephone-like audio and nearly real-time video transmissions at, for example, 15-30 frames per second. In addition to high speed, because the telehealth system involves the transmission of protected health information, the network also addresses privacy and security issues, by using secure connections and secured stations so that only the physician or those under the direction of the physician view the session.

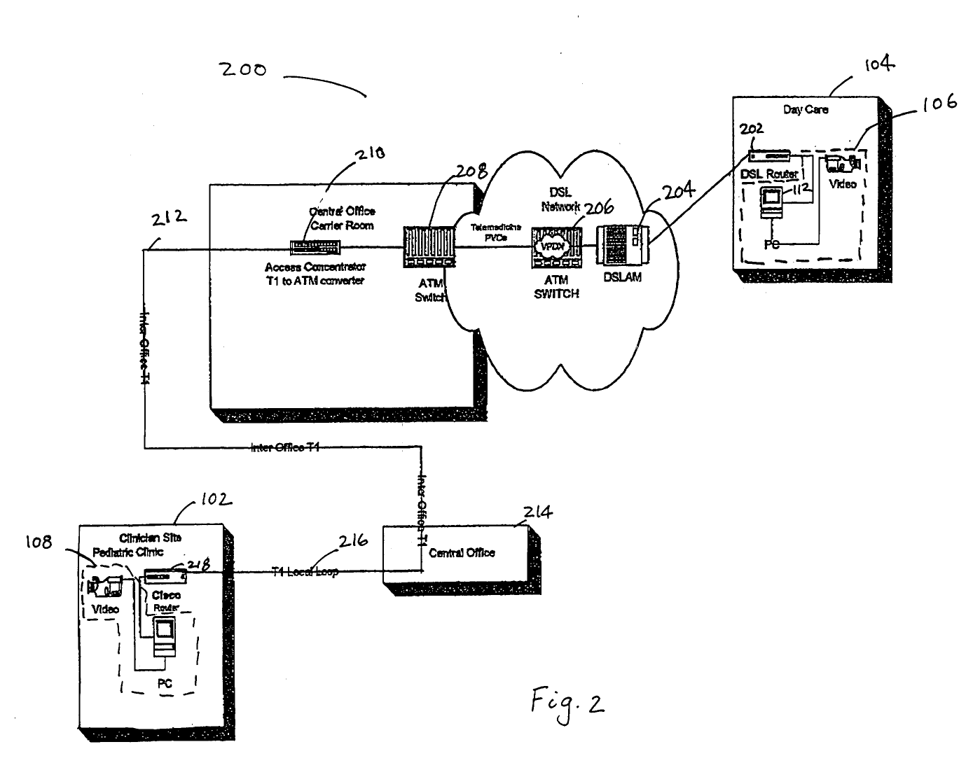

FIG. 2 is a block diagram showing an embodiment of a network model 200 connecting a day care center 104 and a clinician site 102 in accordance with the present invention. The network model depicted in FIG. 2 is one example of a high-speed network connection between a day care center and a clinician. This example is used to illustrate an example of a high-speed networks and is not meant to limit the scope of the embodiments of the present invention. At the day care site 104, the computer 112 at the examination station 106 is connected with a router 202 such as, for example, a digital subscriber line (DSL) router. Router 202 is in turn coupled with a multiplexer device 204 such as, for example, a digital subscriber line access multiplexer (DSLAM), to connect from the examination station 106 to a high speed network via a network device such as, for example, an asynchronous transfer mode (ATM) switch 206. The ATM switch 206 communicates the data over a private virtual circuit (PVC), which is a secure path connecting the ATM switch 206 with another ATM switch 208 at a central office carrier room. ATM switch is in turn coupled to an access concentrator 210, which converts ATM mode data to a T1 (e.g. a high-speed circuit communicating at approximately 1.5 Mbps) protocol. The converted data is transferred over an inter office Ti connection 212 to a central office 214. A T1 local loop 216 connects the central office 214 with the clinician's diagnostic station 108 computer, via a router 218. This network model has the advantages of security as well as requiring a lesser amount of owned equipment as compared to a T1 point to point network. In addition this network model by using a combination of T1 and DSL equipment is more versatile than a purely DSL-based network.

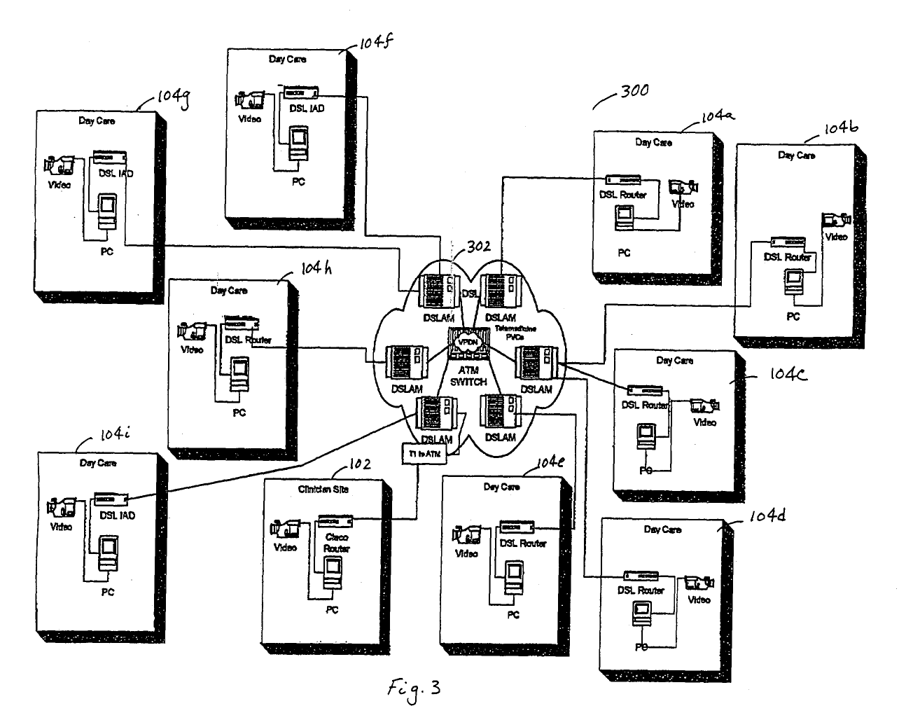

FIG. 3 is a block diagram of an embodiment of an expanded version of the telehealth system of FIG. 2. This figures (FIG. 3) shows a plurality of remote childcare center 104a-i communicating with a clinician site 102 via a network structure 302.

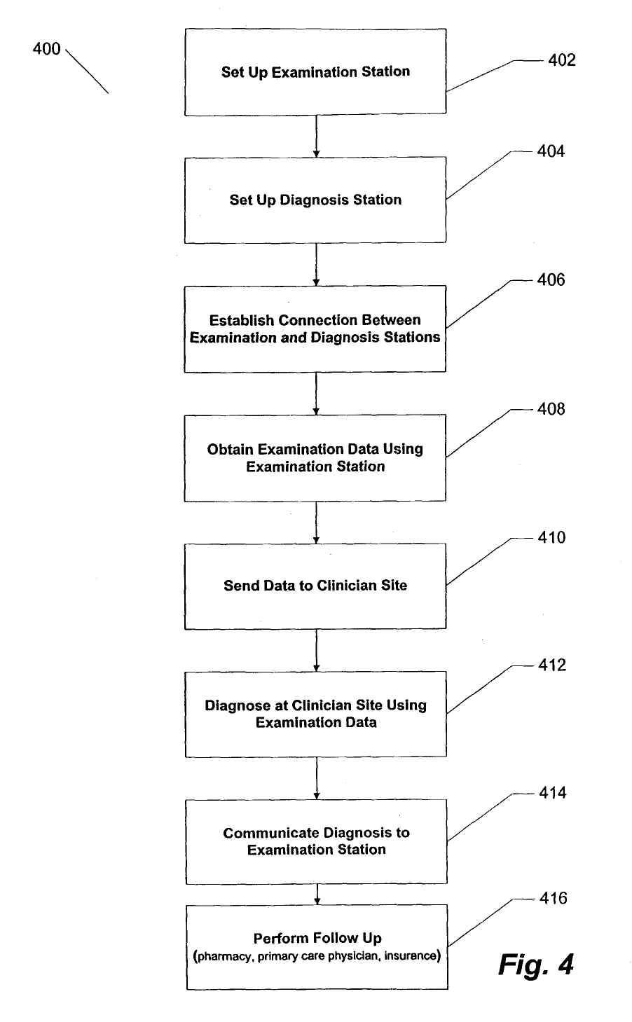

FIG. 4 is a block diagram of an embodiment of a method 400 for the provision of health care services by a health care provider at a remote location to a patient at a childcare center, in accordance with the present invention. First, an examination station 106 and a diagnosis station 108 are set up (steps 402 and 404). Next, a connection between the examination station and the diagnosis station in established (step 406). Next, a telehealth assistant accompanies a patient, for example, a pediatric patient, to the examination station. After confirming a connection between the examination and diagnostic stations, the telehealth assistant obtains examination data using the equipment available at the examination station. Such examination data is obtained using, for example, the stethoscope, the otoscope and other available equipment (step 408). The examination data is next sent to the clinician's site using the network connection (step 410). Using the received data, which includes video or still images, as well as sounds acquired by the stethoscope obtained at the examination station, the health care provider (e.g. a doctor) communicates further with the telehealth assistant and the patient via the cameras and microphones. The further communication may include the asking of routine questions and/or requiring a better reading using the stethoscope or the otoscope, etc. Using the received information, the doctor makes a diagnosis (step 412) and communicates the diagnosis to the telehealth assistant at the remote site (step 414). Additionally certain follow up tasks may also be included (step 416). Such tasks may involve the calling in of a prescription for the treatment of the patient, as well as the reporting of the virtual doctor's visit and diagnosis to the patient's primary health care provider and/or the insurance provider.

Using the method 400 of FIG. 4 in conjunction with the system 100 of FIG. 1, provides highly accessible health services that have been designed to allow clinicians to make the diagnostic and treatment decisions needed to manage acute illness that arises frequently among children in childcare. Both real-time interactive and store-and-forward telemedicine is practiced. Guided by the structured format of forms presented by computer software tailored for the embodiments of the present invention, trained childcare personnel (telehealth assistants) collect information about the present illness and past medical history. Telehealth assistants send completed forms, still images, video clips, and audio files directly to clinicians in remote locations. These store-and-forward “feeds” include digitized input from an electronic stethoscope, an ear-nose-throat endoscope, and an all-purpose digital camera that captures skin and eye lesions as well as respiratory movements and patterns of behavior. Store-and-forward material is supplemented as required by the clinician with real-time close-up video, and additional history obtained by the clinician through a video conference interaction or telephone conversation. Supplemental history may be obtained from other childcare staff or the parent as well as from the telehealth assistant. When prescription medications are indicated, clinicians call in prescriptions to a pharmacy chosen by the family. Prescriptions are delivered to the childcare center by a pharmacy.

Certain aspects of the embodiments of the methods described above may be implemented as a software program or programs. Such a software program may be written using a variety of programming languages, including C, C++, visual C, Java, visual Java, and other languages as is known to those of skill in the art of computer-based and peripheral communication programming.

Certain aspects of the embodiments of the methods described above may be practiced in a multitude of different ways (i.e., software, hardware, or a combination of both) and in a variety of systems. In one embodiment, certain aspects of the described method can be implemented as a software program. The software program may be configured for execution by various computer systems or processors such as, for example, personal computers (e.g. PC's and Macs).

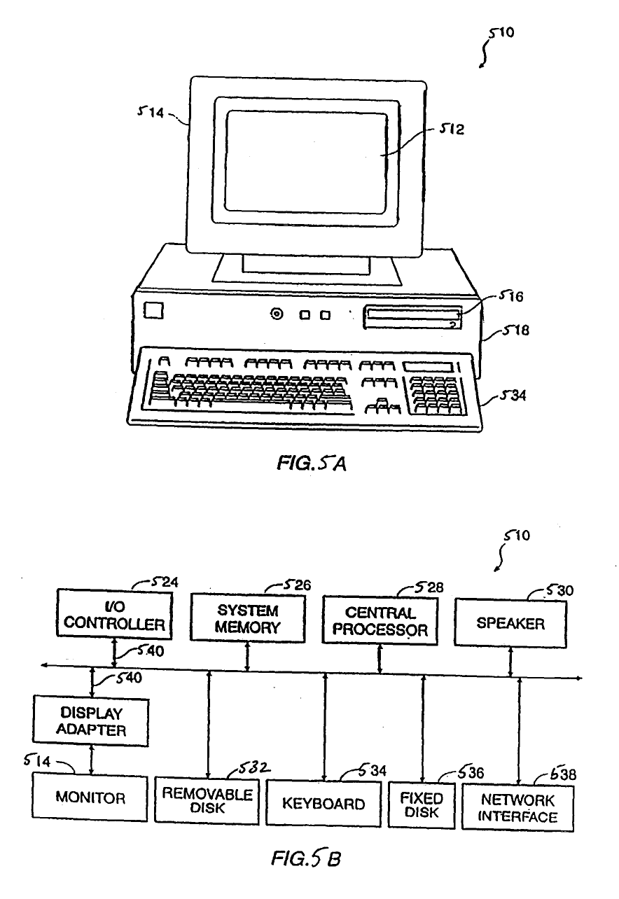

FIG. 5A is diagram of one embodiment of a computer system 510 for executing a software program incorporating aspects of the described method of FIG. 4. Computer system 510 includes a monitor 514, screen 512, cabinet 518, and keyboard 534. A mouse, light pen, a joy stick, a microphone, a video camera, speakers or other I/O interfaces, such as virtual reality interfaces, and the telemedicine tool kit may also be included (not shown) for providing I/O commands. Cabinet 518 houses a CD-ROM drive 516, a hard drive (not shown) or other storage data mediums which may be utilized to store and retrieve digital data and software programs incorporating the present method, and the like. Although CD-ROM 516 is shown as the removable media, other removable tangible media including floppy disks, tape or flash memory may be utilized. Cabinet 518 also houses familiar computer components (not shown) such as a processor, memory, and the like.

FIG. 5B illustrates a simplified system block diagram of a typical computer system used to execute a software program incorporating aspects of the described method for the communication of examination and diagnosis data between computers at the examination and diagnosis stations in accordance with the present invention. As shown in FIGS. 5A and 5B, computer system 510 includes monitor 514 which optionally is interactive with the I/O controller 524. Computer system 510 further includes subsystems such as system memory 526, central processor 528, speaker 530, removable disk 532, keyboard 534, fixed disk 536, and network interface 538. Other computer systems suitable for use with the described method may include additional or fewer subsystems. For example, another computer system could include more than one processor 528 (i.e., a multi-processor system) for processing the digital data. Arrows such as 540 represent the system bus architecture of computer system 510. However, these arrows 540 are illustrative of any interconnection scheme serving to link the subsystems. For example, a local bus could be utilized to connect the central processor 528 to the system memory 526. Computer system 510 shown in FIG. 5B is but an example of a computer system suitable for use with the present invention. Other configurations of subsystems suitable for use with the present invention will be readily apparent to one of ordinary skill in the art.

Embodiments of the present invention offer several advantages. One advantages is that the system and method bring health services to childcare programs, where the majority of preschool children spend most of their time while their parents work. The system and method reduces several problems stemming from illness among children in childcare centers. For example, the invention reduces loss of parent time at work, by not requiring a parent to leave his or her work place to pick up and take the child to see a doctor. Also, the invention reduces the use of expensive health services, such as emergency rooms or after hour clinic visits to address problems that can be managed more efficiently via telemedicine. One reason that the embodiments of the present invention enable an efficient management of problems is that the entire process of examination and diagnosis can take minutes instead of hours, which are usually spent getting to and waiting around before seeing a doctor. Furthermore, additional exposures are reduced by obviating the need to move a potentially sick child to a doctor's waiting room that is full of other sick children. The embodiments of the invention also provide a competitive advantage to childcare centers that provide access to such a telemedicine system, since parents will tend to chose a center that provides such a system.

Additional advantages include better examinations and diagnoses because, for example, physicians can get a better look inside a child's ear via telemedicine, because a doctor can freeze the image obtained by a digital otoscope on the monitor and examine it closely. During a typical doctor's visit, a child might move around so much that it's hard to get a good look inside the ear, especially for toddler and other pediatric patients. Embodiments of the present invention are also advantageous because young children are able to sit still while the day care telehealth assistant examines them, since they enjoy hearing themselves on the speakers and watching themselves on the monitor. Likewise, a particular sound, such as the sound of a cough or a gurgle may be recorded once and played back repeatedly, thus allowing for a more effective evaluation by the physician.

The utility, reliability and efficacy of the embodiments of the telemedicine model in accordance with embodiments of the present invention for common childhood illnesses have been established by the inventors as a part of several studies. In one such study, illness visits to a hospital-based primary care facility in Rochester, N.Y., received duplicate exams by experienced pediatricians. Visits were randomly assigned to receive either a telemedicine or in-person duplicate exam. Eligibility criteria included initial visit for an illness and excluded problems inappropriate for our telemedicine model; e.g., needing skilled palpation or x-rays. The study set out to show that when diagnoses of both telemedicine duplicate providers and in-person duplicate providers are compared with diagnoses of regular providers in a pediatric ambulatory setting, agreement for telemedicine will equal agreement for in-person exams. In this study, the five physicians conducting duplicate exams were all experienced, attending-level pediatricians with a primary clinical base in either the emergency department or the primary care center where the study was conducted. On the date the study was initiated, time since completion of pediatric residency for this group averaged 13.7 years (range: 8.7-24.7). Duplicate exams were conducted with the patient in regular exam rooms in as unobtrusive a manner as possible, either before or after the regular provider. The remote site, where duplicate providers conducted telemedicine exams, was a separate room in the primary care center. Duplicate providers waited there for instructions to see patients who had been assigned randomly to either telemedicine or in-person duplicate exams. A research assistant with no prior medical or nursing training served as the telemedicine assistant, rolling a mobile patient-site telemedicine unit one exam room to another as needed to allow telemedicine exams. Connection to the medical center's broadband telecommunications network, which served the remote telemedicine clinician site, was maintained with wireless technology, obviating the need to re-establish a connection as the patient-site telemedicine unit moved between exam rooms. The study involved a trained telemedicine assistant at a remote site; real-time video and audio interaction with a child, parent and telemedicine assistant; several computer-driven remote sensing devices including an ear-nose-throat endoscope, an electronic stethoscope, and a digital camera; and simple office procedures (vital signs, oximetry, cerumen removal, palpation for lymph nodes, administration of nebulized medication); and simple laboratory studies (rapid strep test, urine dip stick).

The study showed that among the 492 valid cases, 54 cases (11.0%) had a disagreement on primary diagnosis. Disagreement on primary diagnosis was marginally more common among cases randomized to telemedicine than among those randomized to in-person (13.8% vs. 8.3%, χ²=3.82, P=0.051). This difference was due to disagreements in which only one duplicate provider diagnosed acute otitis media. From this study it can be concluded that use of telemedicine in accordance with the embodiments of the present invention to enhance access to care for common, acute childhood illness would lead to evaluation and child outcomes that are very similar to those with in-person care. Because telemedicine provides images for deliberate evaluation, and in-person tympanic membrane exams often involve only a fleeting glance, the advantage of in-person exams on agreement about acute otitis media is attributable to a disadvantage in accuracy, shared with the regular provider, when compared to telemedicine. Findings that reliability of telemedicine is very similar to that of in-person care strongly supports the position that use of telemedicine to enhance access to care for common, acute childhood illness allows high quality care, with comparable diagnosis and child health outcomes, while enhancing access tremendously. Furthermore, the clinical experience with telemedicine suggests that diagnostic accuracy can be enhanced not only by examining a fixed image, but by electronic adjustment of image characteristics (e.g., brightness, contrast, color balance, hue, saturation) and through the use of video clips. Video clips can be played back repeatedly, and they can be “frozen” on frames with the best combination of lighting, focus, and anatomic field of vision is achieved. Often, frames providing the highest quality diagnostic information equate to just a fleeting glance in real-time. The hypothesis of telemedicine superiority, especially for ear exams is worth noting. Providing both video clips and still images of tympanic membranes to an expert panel would provide an operational “gold standard” with a high level of validity.

Additionally, results of experience with the three experimental pilot centers show that among the 804 children who were enrolled in one of these centers at any time following initiation of telehealth services in accordance with embodiments of the present invention, and whose parents provided consent for tracking of absence, illness-related absence and illness-related health services utilization, 732 (91.0%) also provided consent for their children to receive telehealth services. For 3.6% of the 575 telehealth visits provided, the telehealth clinician (physician or nurse practitioner) has recommended contact, either the same day or following day, with the primary care provider. This suggests that the telehealth clinician was able to manage the problem completely for 96.4% of visits. Furthermore, an observation has been the decrease in absence from childcare due to illness following the introduction of a telehealth system in accordance with embodiments of the present invention. Prior to the telehealth system in accordance with embodiments of the present invention, rates of absence due to illness during high-illness and low-illness periods have averaged 2.10 and 0.75 per 100 enrolled children per week, respectively. Following the introduction of telehealth, rates of absence due to illness during high-illness and low-illness periods have averaged 0.45 and 0.37 per 100 children per week. Post-telehealth rates represent reductions of 78.4% and 51.4%.

While in describing the embodiments of the present invention, a childcare center has been described as the site for the examination station 104, it is understood that the examination station 104 may be located at any location where a patient is located and which is remotely located with respect to the diagnosis station 102, where a clinician is located. Furthermore, the term remote as used herein, refers to an examination station that is not in the same location as the diagnosis station, and therefore, remotely located can include on the other side of the same room, in another room, in a room on the other side of a building, across town, across the country, or across the globe, etc.

As will be understood by those of skill in the art, the present invention may be embodied in other specific forms without departing from the essential characteristics thereof. For example, an examination station may be placed at any location other than a day care center, or the diagnosis station may be placed at any location other than a clinic such as, for example, a doctor's home office. Moreover, the high-speed network can also be extended to enable wireless connectivity, such that a doctor equipped with a properly configured laptop or compact computer, is enabled to diagnose a patient from virtually any location in the world. Accordingly, the foregoing is intended to be illustrative, but not limiting of the scope of the invention, which is set forth in the following claims.

What is claimed is:

1. A system for providing medical evaluation and treatment recommendations for children at a childcare center, comprising:
an examination station for medically examining a patient, located at the childcare center, wherein said examination station comprises:
an examination computer,
a camera coupled with said examination computer,
a diagnostic tool set coupled with said examination computer;
a diagnosis station for diagnosing the condition of the patient, remotely located from the examination station; wherein said diagnosis station comprises:
a diagnosis computer,
a camera coupled with said diagnosis computer;
modules for interfacing said diagnosis computer with said examination computer; and
a communication system for coupling said examination station with said diagnosis station.

2. The system of claim 1 wherein said examination station further comprises a microphone coupled with said examination computer.

3. The system of claim 1 wherein said examination station further comprises a speaker coupled with said examination computer.

4. The system of claim 1 wherein said diagnostic tool set comprises a diagnostic tool selected from the group consisting of a stethoscope, an endoscope, an otoscope, and combinations thereof.

5. The system of claim 1 wherein said diagnosis station further comprises a microphone coupled with said diagnosis computer.

6. The system of claim 1 wherein said diagnosis station further comprises a speaker coupled with said diagnosis computer.

7. The system of claim 1 wherein said modules for interfacing said diagnosis computer with said examination computer comprise modules for transferring audio data between said diagnosis computer and said examination computer.

8. The system of claim 1 wherein said modules for interfacing said diagnosis computer with said examination computer comprise modules for transferring image data between said diagnosis computer and said examination computer.

9. The system of claim 1 wherein said modules for interfacing said diagnosis computer with said examination computer comprise modules for transferring real-time data between said diagnosis computer and said examination computer.

10. The system of claim 1 wherein said modules for interfacing said diagnosis computer with said examination computer comprise modules for storing and forwarding data between said diagnosis computer and said examination computer.

11. The system of claim 1 wherein said communication system comprises a server configured for secured communication between said examination station and said diagnosis station.

12. The system of claim 1 wherein said communication system comprises a wireless connection between said examination station and said diagnosis station.

13. A method for providing medical evaluation and treatment recommendations for children at a childcare center, comprising:
providing an examination station at a childcare center;
providing a diagnosis station at a location different from said examination station;
providing a communication interface between said examination station and said diagnosis station;
examining a patient using said examination station to generate examination data;
transmitting said examination data to said diagnosis station;
evaluating the condition of said patient using said diagnosis station to generate evaluation data; and
communicating said evaluation data from said diagnosis station to said examination station using said interface.

14. The method of claim 13 where said providing an examination station at a childcare center, comprises:
providing an examination computer,
providing a camera coupled with said examination computer; and
providing a diagnostic tool set coupled with said examination computer.

15. The method of claim 14 further comprising providing a microphone coupled with said examination computer.

16. The method of claim 14 further comprising providing a speaker coupled with said examination computer.

17. The method of claim 14 wherein said providing a diagnostic tool set coupled with said examination computer comprises providing a diagnostic tool selected from the group consisting of a stethoscope, an endoscope, an otoscope, and combinations thereof.

18. The method of claim 13 where said examining is conducted by a trained telehealth assistant in communication with a clinician, where the clinician is located adjacent to the diagnosis station.

19. The method of claim 13 where said transmitting said examination data comprises transmitting audio, or image data from said examination station to said diagnosis station.

20. The method of claim 19 wherein said transmitting comprises transmitting said data in real-time.

21. The method of claim 13 further comprising diagnosing a condition consisting of colds, fevers, ear infections, pink-eye, impetigo and combinations thereof for pediatric patients.
